(19) United States
(12) Plant Patent Application Publication
(10) Pub. No.: US 2021/0219479 P1
(43) Pub. Date: Jul. 15, 2021

(54) AGAPANTHUS PLANT NAMED 'LITTLE GALAXY

(50) ;

(71) Applicant: Hans A. Hansen, Zeeland, MI (US)

(72) Inventor: Hans A. Hansen, Zeeland, MI (US)

(73) Assignee: Walters Gardens Inc., Zeeland, MI (US)

(21) Appl. No.: 16/873,015

(22) Filed: Jan. 15, 2020

(51) Int. Cl. A01H 6/56

(52) U.S. Cl. PLT/398

(57) ABSTRACT

A new and distinct Agapanthus plant named ‘Little Galaxy’ characterized by winter-hardy, very compact habit with clean, lanceolate, medium-green foliage that goes dormant in the Michigan winter; single medium blue flowers on numerous, short stems producing rounded globes of flowering beginning about mid-July and with repeating new scapes into mid-September for about eight weeks.

Botanical classification: Agapanthus hybrid.

Variety denomination: ‘Little Galaxy’.

### STATEMENT REGARDING PRIOR DISCLOSURES UNDER 37 CFR 1.77(b)(6)

The first public disclosure of the claimed plant, in the form of a website to the public and email release to customers, was made by Walters Gardens, Inc. on Feb. 1, 2019. Since then Walters Gardens, Inc. printed a description and photograph in the “Walters Gardens 19-20 Catalog” released on May 29, 2019 and began selling it to customers on Jan. 6, 2020. Walters Gardens, Inc. obtained the new plant and information about the new plant directly from the inventor. No plants of Agapanthus ‘Little Galaxy’ have been sold, in this country or anywhere in the world, nor has any disclosure of the new plant been made, more than one year prior the filing date of this application, and such sale or disclosure within one year was either derived directly or indirectly from the inventor.

### BACKGROUND AND ORIGIN OF THE PLANT

The present invention relates to a new and distinct Lily of the Nile plant, Agapanthus ‘Little Galaxy’ hereinafter also referred to as the new plant or just the cultivar name, ‘Little Galaxy’. Agapanthus ‘Little Galaxy’ was selected by the inventor in July of 2012 in a trial field at a wholesale perennial nursery in Zeeland, Mich., USA. The new plant originated from a controlled cross conducted by the inventor with the specific intention to improve the garden worthiness, reduce plant size, expand color regimens and increase flowering period which were some of the criteria of further trials in the trial beds at the same nursery in Zeeland, Mich. The female or seed parent was Agapanthus campanulatus subspecies patens ‘Kingston Blue’ (not patented) and the male or pollen parent was ‘Prolific Blue’ (not patented) crossed on Jul. 12, 2010. The new plant was selected as a single seedling from this cross and after confidential evaluations in a trial bed beginning in 2012 in Zeeland, Mich. and was assigned the breeder code H10-06-01 through the remaining trial period.

The new plant has been asexually propagated by division and shoot tip tissue culture at the same wholesale nursery in Zeeland, Mich. since 2014 with all resultant asexually propagated plants having retained all the same unique traits as the original plant. Agapanthus ‘Little Galaxy’ has proven to be stable and reproduces true to type in successive generations of asexual reproduction.

The most similar known Lily of the Nile cultivars are: the female parent ‘Kingston Blue’, male parent ‘Prolific Blue’ and ‘Lapis Lazuli’ (not patented). ‘Kingston Blue’ is taller in habit and less winter hardy, and has flowers that have a more bluish-purple coloration. ‘Prolific Blue’ is equally winter hardy, has a taller in habit and has a more grayed-blue flower color. ‘Lapis Lazuli’ is taller in habit with fewer flowers per pseudo-umbel. ‘Galaxy Blue’ U.S. Plant Pat. No. 31,421 is equally winter hardy, has a much taller habit and the flowers tend to have slightly more purplish tint that lighten with maturity.

Agapanthus ‘Little Galaxy’ differs from all other Lily of the Nile plants known to the applicant, by the combination of the following traits:
1. Medium blue flowers with darker blue center veins;
2. Short overall habit with numerous flower stems;
3. Long season of bloom.
4. Winter-hardy to USDA zone 6, compact, clean, medium-green foliage that is deciduous and goes dormant in the winter;
5. Flowering begins about mid-July with excellent coverage and sending new scapes into mid-September for about five to eight weeks;

### BRIEF DESCRIPTION OF THE DRAWINGS

The photograph of the new plant demonstrates the overall appearance of the plant, including the unique traits. The colors are as accurate as reasonably possible with color reproductions. Ambient light spectrum, temperature, source and direction may cause the appearance of minor variation in color.

FIG. 1 shows an eight-year-old plant in peak flower during mid-summer in a full-sun trial garden in Zeeland, Mich.

FIG. 2 shows a close-up of the flower and buds.

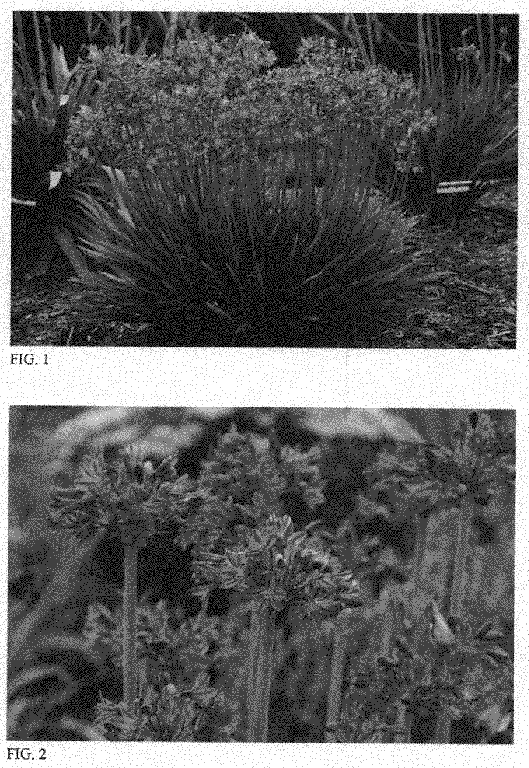

### DETAILED BOTANICAL DESCRIPTION

The following descriptions and color references are based on the 2015 edition of The Royal Horticultural Society Colour Chart except where common dictionary terms are used. The new plant, Agapanthus ‘Little Galaxy’, has not been observed under all possible environments. The phenotype may vary slightly with different environmental conditions, such as temperature, light, fertility, moisture and maturity levels, but without any change in the genotype. The following observations and size descriptions are of an eight-year-old plant in a sunny research garden bed in Zeeland, Mich. with and supplemental water and fertilizer.
Botanical classification: Agapanthus hybrid;
Parentage: Female or seed parent is A. campanulatus ‘Kingston Blue’; male or pollen parent is A. [symbol] ‘Prolific Blue’;
Propagation: Division of the rhizome and shoot tip tissue culture;
Growth rate: Moderate;
Crop time: About 12 to 15 weeks to flower starting in spring in a 3.8 liter container from vernalized one-year-old plant;
Rooting habit: Primary roots thick and fleshy, secondary roots fibrous;
Root color: Nearest RHS 159C depending on soil type;
Plant shape and habit: Winter-hardy herbaceous perennial with pseudo-umbel inflorescence; about 95 flowering stems surrounded by acaulescent foliage forming a dense mound;
Plant size: Foliage height about 32.0 cm tall from soil line to the top of the leaves, about 66.0 cm tall to highest inflorescence and about 80.0 cm wide at the widest point at the top of the inflorescences;
Leaves: Linear; acaulescent; deciduous: bi-ranked; entire; glabrous and glaucous both abaxial and adaxial; sessile; apex acute, base sheathing scape; to about 39.0 cm long and 8.0 mm across, average about 28.0 cm long and about 8.0 mm across; about 12 per division; attitude upright becoming outwardly and arching;
Leaf color: Abaxial and adaxial base nearest RHS NN155D; mature and young adaxial nearest RHS N138A and abaxial nearest RHS N138C;
Veins: Parallel; not significant; abaxial midrib costate, about 2.0 mm across and color same as surrounding leaf tissue;
Inflorescence: Pseudo-umbel initially sheathed in two bracts; broad oblate; about 95 cm across and 75 cm tall; flowers opening from the sides first and then toward the center;
Bracts: Deltoid; dehiscent; medium-length acuminate apex; truncate base; scarious; to about 12.0 mm across near middle and 32.0 mm long;
Bract color: Variable; nearest RHS 165D and RHS 165B; anthocyanin coloration absent;
Flowers: Funnelform; single, with two sets of three tepals; about 40 to 75 per scape; upward and outwardly facing; about 30.0 mm across and 22.0 mm deep; individually lasting for about four to five days, individual inflorescence lasting about two weeks; flowers remain effective from late-July repeating into mid-September for approximately five to eight weeks in Zeeland, Mich.;
Flower fragrance: None detected;
Buds one to two days prior to opening: Ellipsoidal with rounded apex and base; about 16.0 mm long and about 6.0 mm in diameter at widest point with near apex;
Bud color one day from opening: Apical and along tepal margins nearest RHS 96A, slightly lighter proximally nearest RHS 96C toward middle with base nearest RHS 96B;
Tepals: 2 sets of 3; both sets identical; open to nearly flat face; perianth tube length short;
Inner tepals: Glabrous; obtuse; rounded apex; base attenuate, fused in proximal 8.0 mm; margin entire; width at base about 2.0 mm; about 9.0 mm across at widest point and about 22.0 mm long;
Outer tepals: Glabrous; obtuse; rounded apex; base attenuate, fused in proximal 8.0 mm; margin entire; width at base about 2.0 mm; about 7.0 mm across at widest point and about 22.0 mm long;
Tepal color adaxial: Both sets identical; distally between RHS 97A and RHS 97B with midrib nearest RHS 96A, basal 4.0 mm lighter than RHS 97D;
Tepal color abaxial: Both sets identical; nearest RHS 97B; base and midrib between RHS 99A and RHS 96A;
Pedicel: Cylindrical; average about 30.0 mm long and 1.0 mm diameter; upright and outwardly;
Pedicel color: Nearest RHS 146B blushed with nearest RHS 187A;
Peduncle: Glabrous; becoming terete, hollow in maturity; usually one per division; erect; to about 6.0 mm diameter at base, average 63.0 cm tall; extending above foliage;
Peduncle color: Nearest RHS 145A proximally, distally between RHS 145A and RHS 146D, center portion between RHS 146B and RHS 146C; anthocyanin coloration absent;
Gynoecium: Single; tricarpelled; about 18.0 mm long;
   Style.—Single, about 9.0 mm long, 0.7 mm diameter; color nearest RHS NN155D.
   Stigma.—About 0.3 mm across; color nearest RHS NN155D.
   Ovary.—Superior; ellipsoidal; about 6.0 mm long and 3.0 mm diameter near middle with an acute apex and truncate base; color nearest RHS 145C.
Androecium: Six;
   Filaments.—Six; adnate to inner corolla in proximal 6.0 mm and free in distal 10.0 mm; about 0.5 mm in diameter; arcuate slightly upward distally; moderately exserted beyond corolla; color between RHS 95D and RHS 92D.
   Anthers.—Oblong; basifixed, longitudinal; about 3.0 mm long and 1.0 mm wide; color nearest RHS 103A.
   Pollen.—Color nearest RHS 151D.
Fruit: Oblong ellipsoidal; non-fleshy, dehiscent, tri-loculicidal capsule with three distinct lobes; about 20.0 mm long and 7.0 mm in across; color while maturing nearest RHS N144D and at dehiscence variable between RHS 164C and RHS 1658;
Seed: Up to about 18 per capsule; flattened single wing with embryo situated near one end; about 8.0 mm long, about 3.0 mm wide and about 1.0 mm thick at embryo; color nearest RHS 202A;
Disease and pest resistance and tolerance: ‘Little Galaxy’ shows typical Lily of the Nile resistant to deer and rabbits but has not shown resistance to diseases and pests beyond that common for Lily of the Nile plants. The plant grows best and shows best coloration with plenty of moisture, adequate drainage, but is able to tolerate some drought when mature and direct sun without leaf burn when provided sufficient water.

Hardiness at least from USDA zone 6 through 11. The new plant is useful for landscaping en masse, as a single specimen or small groups, as a container plant or as a cut flower.

I claim:

1. A new and distinct ornamental Agapanthus plant named ‘Little Galaxy’ as herein described and illustrated.
